(19) United States
(12) Patent Application Publication
(10) Pub. No.: US 2017/0020880 A1
(43) Pub. Date: Jan. 26, 2017

(54) PHARMACEUTICAL COMPOSITIONS OF TRAMETINIB

(71) Applicant: HETERO RESEARCH FOUNDATION, HYDERABAD (IN)

(72) Inventors: BANDI PARTHASARADHI REDDY, HYDERABAD (IN); PODILE KHADGAPATHI, HYDERABAD (IN); DEVIPRASAD SUNIL TIWARI, HYDERABAD (IN); KALIDINDI ASHOK KUMAR, HYDERABAD (IN)

(21) Appl. No.: 15/209,986

(22) Filed: Jul. 14, 2016

(30) Foreign Application Priority Data
Jul. 22, 2015 (IN) 3772/CHE/2015

(51) Int. Cl. A61K 31/519; A61K 9/20; A61K 9/28

(52) U.S. Cl. CPC A61K 31/519; A61K 9/28; A61K 9/2893; A61K 9/2013; A61K 9/2031

(57) ABSTRACT

The present disclosure relates to pharmaceutical compositions comprising trametinib. More specifically, the present disclosure relates to solid compositions comprising trametinib and processes for preparing the same.

### CROSS-REFERENCE TO RELATED APPLICATIONS

This patent application claims priority to Indian patent application number 3772/CHE/2015, filed on Jul. 22, 2015, the contents of which are incorporated by reference herein in their entirety.

### FIELD OF THE DISCLOSURE

The present disclosure relates to pharmaceutical compositions comprising trametinib. More particularly, the present disclosure relates to solid oral compositions comprising trametinib and processes for preparing the same.

### BACKGROUND

Trametinib is a kinase inhibitor. It is chemically described as N-(3-{3-Cyclopropyl-5-[(2-fluoro-4-iodophenyl)amino]-6,8-dimethyl-2,4,7-trioxo-3,4,6,7-tetrahydropyrido [4,3-d]pyrimidin-1(2H)-yl}phenyl)acetamide. Its empirical formula is C₂₆H₂₃FIN₅O₄ and it has the following structural formula:

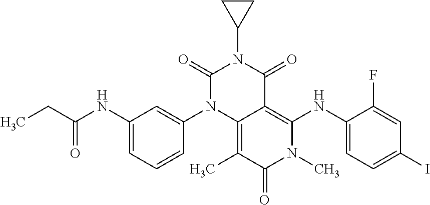 [SMILES: CCC(=O)Nc1cccc(-n2c(=O)n(C3CC3)c(=O)c3c(Nc4ccc(I)cc4F)n(C)c(=O)c(C)c32)c1]

The U.S. FDA has approved trametinib as 0.5 mg, 1 mg and 2 mg base equivalent tablets under the brand name MEKINIST® to Novartis.

U.S. Pat. No. 7,378,423 discloses trametinib or its pharmaceutically acceptable salt, hydrate, or solvate thereof.

U.S. Pat. No. 8,580,304 discloses tablet compositions comprising trametinib dimethyl sulfoxide solvate and 25% to about 89% of excipients.

U.S. Pat. No. 9,155,706 discloses tablet compositions comprising trametinib dimethyl sulfoxide solvate having a particle size of 30 microns or less.

There exists a need to develop alternative formulations of trametinib with improved dissolution and bioavailability. Accordingly, the inventors of the present invention have developed trametinib compositions using alternative formulation techniques to improve the dissolution properties of the drug and thus solubility and bioavailability.

### SUMMARY

The present disclosure relates to pharmaceutical compositions comprising trametinib, and more particularly to solid oral compositions of trametinib.

One embodiment of the present disclosure is a tablet composition comprising a) trametinib or its pharmaceutically acceptable salt or solvate, and b) greater than or equal to 90 wt % of pharmaceutically acceptable excipients, based on the total weight of the composition.

One another embodiment of the present disclosure is a tablet composition comprising a) trametinib or its pharmaceutically acceptable salt or solvate, and b) one or more solubility enhancers that is lipoamino acid, a poloxamer or both.

Yet another embodiment of the present disclosure is a process comprising a) dispersing trametinib or its pharmaceutically acceptable salt or solvate in one or more non-aqueous solvents to provide a dispersion, b) sifting and blending one or more intragranular excipients to provide a blend, c) granulating the blend of step (b) using the dispersion of step (a) to provide granules, d) drying the granules obtained in step (c) to provide dried granules, e) sifting one or more extragranular excipients and blending the sifted extragranular excipients with the dried granules of step (d) to provide blended granules, f) compressing the blended granules of step (e) into tablets, and g) optionally film coating the tablets obtained in step (f). Also included is the product of the foregoing process.

A further embodiment is related to a method of treating unresectable or metastatic melanoma with BRAF V600E or V600K mutations in a patient in need thereof by administering the compositions of the present disclosure.

### DETAILED DESCRIPTION

The present disclosure is related to pharmaceutical compositions comprising trametinib, and more particularly to solid oral compositions of trametinib.

The term “active agent” as used herein refers to “trametinib”, wherein trametinib includes trametinib in the form of its free base or a pharmaceutically acceptable salt or solvate thereof. More specifically trametinib is trametinib dimethyl sulfoxide solvate.

The term “excipient” means a pharmacologically inactive component such as a diluent, a binder, a disintegrant, a glidant, a lubricant, and the like, of a pharmaceutical product. The excipients that are useful in preparing a pharmaceutical composition are generally safe, non-toxic and are acceptable for veterinary as well as human pharmaceutical use. Reference to an excipient includes both one and more than one such excipient.

The term “composition” or “pharmaceutical composition” or “solid oral composition” or “dosage form” as used herein synonymously include solid dosage forms such as tablets, capsules, granules, pellets and the like meant for oral administration. More specifically a pharmaceutical dosage form is a tablet.

Compositions according to the present disclosure are in the form of a tablet, capsule, granules, or pellets, preferably a tablet.

One embodiment of the present disclosure is a tablet composition comprising a) trametinib or its pharmaceutically acceptable salt or solvate, and b) greater than or equal to 90 wt % of pharmaceutically acceptable excipients, based on the total weight of the composition.

According to the present disclosure, pharmaceutically acceptable excipients are selected from diluents, binders, disintegrants, solubility enhancers, lubricants, glidants and combinations thereof.

Another embodiment of the present disclosure is a tablet composition comprising: a) trametinib or its pharmaceutically acceptable salt or solvate, and b) one or more solubility enhancers that is lipoamino acid, a poloxamer, or both.

Diluents include, but are not limited to, mannitol, lactose, microcrystalline cellulose, starch, corn starch, pregelatinized starch, maize starch, potato starch, powdered celluloses, sorbitol, xylitol, dibasic calcium phosphate, calcium phosphate, calcium carbonate, magnesium carbonate, and the like, and combinations thereof.

Binders include, but are not limited to, polyvinyl pyrrolidone, copovidone, hydroxypropyl cellulose, hydroxypropyl methylcellulose, pregelatinized starch, powdered acacia, gelatin, guar gum, carbomers, and the like, and combinations thereof.

Disintegrants include, but are not limited to, polacrilin potassium, croscarmellose sodium, sodium starch glycolate, crospovidone, pregelatinized starch, and the like, and combinations thereof.

Solubility enhancers, include but are not limited to, lipoamino acids, sorbitan mono laurate, sodium lauryl sulphate, polyoxyethylene-polyoxypropylene block copolymers (also known as poloxamers), polyethylene glycols, sodium stearyl sulfate, sodium oleyl sulfate, isopropyl myristate, sodium cetyl sulfate, sodium dodecylbenzene sulfonate, dialkyl sodium sulfosuccinates, polysorbates, β-cyclodextrins, and the like, and combinations thereof.

Lipoamino acid is marketed as “SEPICLEAR™ PPI” by SEPPIC.

In yet another embodiment, a composition comprises a solubility enhancer that is lipoaminoacid, a poloxamer or both, in an amount of about 0.1 wt % to about 10 wt % based on total weight of the composition. More specifically, the amount of solubility enhancer is about 0.1 wt % to about 5 wt %.

Lubricants include, but are not limited to, magnesium stearate, stearic acid, talc, palmitic acid, sodium stearyl fumarate, carnauba wax, hydrogenated vegetable oils, mineral oil, polyethylene glycols and the like, and combinations thereof.

Glidants include, but are not limited to, colloidal silicon dioxide, magnesium silicate, magnesium trisilicate, talc, and other forms of silicon dioxide, such as aggregated silicates and hydrated silica, and the like, and combinations thereof.

Tablet compositions according to the present disclosure contain trametinib in an amount of 0.5 mg, 1 mg or 2 mg base equivalent.

Tablet compositions according to the present disclosure are prepared by wet granulation either by aqueous granulation or non-aqueous granulation techniques, preferably by non-aqueous granulation.

Non-aqueous solvents suitable for granulation include one or more organic solvents such as acetonitrile, dichloromethane, ethanol, methanol, acetaldehyde, acetone, benzene, carbon disulphide, carbon tetrachloride, 1,2 dichloroethane, N,N-dimethylformamide, 1,4-dioxane, epichlorhydrin, ethyl acetate, ethyl ether, ethylene glycol, 2-ethoxyethanol (acetate), formaldehyde, isopropanol, methyl n-butyl ketone, methyl ethyl ketone, 2-methoxyethanol (acetate), perchloroethylene, toluene, 1,1,1-trichloroethane, trichloroethylene; and the like, and combinations thereof.

Yet another embodiment of the present disclosure is a process comprising a) dispersing trametinib or its pharmaceutically acceptable salt or solvate in one or more non-aqueous solvents to provide a dispersion, b) sifting and blending one or more intragranular excipients to provide a blend, c) granulating the blend of step (b) using the dispersion of step (a) to provide granules, d) drying the granules obtained in step (c) to provide dried granules, e) sifting one or more extragranular excipients and blending the sifted extragranular excipients with the dried granules of step (d) to provide blended granules, f) compressing the blended granules of step (e) into tablets, and g) optionally film coating the tablets obtained in step (f). Also included is the product of the foregoing process.

The tablets prepared are optionally coated using suitable film forming agents.

A film coat on the tablet provides an elegant appearance, protects the tablet from moisture, and further contributes to the ease with which it can be swallowed.

The solid oral dosage forms of the present disclosure are optionally coated with an aqueous or non aqueous solution comprising film forming polymers and one or more of plasticizers, opacifiers, surfactants, anti-tacking agents, coloring agents, and the like, and combinations thereof.

Coating polymers, include but are not limited to, alkyl celluloses such as, methyl cellulose, ethyl cellulose, hydroxypropyl cellulose, hydroxypropyl methyl cellulose, cellulose acetate, cellulose propionate, carboxymethyl cellulose, polyethyleneglycol, polyethyleneoxide, polyvinylalcohol, polyvinylacetate, poly vinyl chloride, polyvinyl pyrrolidone, and combinations thereof.

Plasticizers include, but are not limited to, glyceryl monostearate, triethyl citrate, macrogols, lactic acid, lactic acid acetamide, sorbitol, glycerin, triacetin, acetyl triethyl citrate, acetyl tributyl citrate, polyvinylpyrrolidone, triethylene glycol, ethylene glycol monooleate, acetylated monoglycerides, cetyl alcohol and other hydrogenated oils and waxes, as well as polyethylene glycol, and the like, and combinations thereof.

Further embodiment relates to a method of treating unresectable or metastatic melanoma with BRAF V600E or V600K mutations in a patient in need thereof by administering compositions of the present disclosure.

Certain specific aspects and embodiments of this invention are described in further detail by the examples below, which are provided only for the purpose of illustration and are not intended to limit the scope of the invention in any manner.

### EXAMPLES

### Examples 1-4

| | Exa | Exa | Exa | |
|---|---|---|---|---|
| Ingredients | Mg/ | Mg/ | Mg/ | Exa |
| Drug dispersion | | | | |
| Trametinib | 2.26 | 2.26 | 2.26 | 2.26 |
| SEPICLEAR ™ ^{#} | — | — | 2.00 | 2.00 |
| Acetonitrile | q.s | — | q.s | — |
| Dichloromethane | q.s | q.s | q.s | q.s |
| Intragranular | | | | |
| Microcrystalline | 52.7 | 52.7 | 52.7 | 52.7 |
| Mannitol | 104. | 104. | 103. | 103. |
| Poloxamer | 1.0 | 1.0 | — | — |
| Croscarmellose | 2.0 | 2.0 | 2.0 | 2.0 |
| Extra granular | | | | |
| Microcrystalline | 10.0 | 10.0 | 10.0 | 10.0 |
| Croscarmellose | 2.0 | 2.0 | 2.0 | 2.0 |
| Magnesium | 1.0 | 1.0 | 1.0 | 1.0 |
| Core tablet weight | 175 | 175 | 175 | 175 |
| Film-coating | 4.37 | 4.37 | 4.37 | 4.37 |
| Total tablet weight | 179. | 179. | 179. | 179. |

*2.26 mg of trametinib DMSO is equivalent to 2 mg of trametinib.
^{#}SEPICLEAR ™ is a pure lipoamino acid.

Brief Manufacturing Process:

a) Trametinib DMSO and SEPICLEAR™ were dispersed in acetonitrile and/or dichloromethane,

b) intragranular excipients were sifted, blended and the resultant blend was granulated with the dispersion of step (a),

c) the granules produced were dried and sifted,

d) extragranular excipients were sifted separately and mixed with the dried granules obtained in step (c),

e) lubricated granules of step (d) were compressed into tablets using suitable tooling.

### Dissolution Studies:

Dissolution testing was performed for the tablets prepared according to Example 1, 2 and 3 using a USP apparatus II, 60 rpm in 500 ml of medium of 4.5 acetate buffer and 0.75% SLS.

TABLE 1

| | Time in Minutes | | | |
|---|---|---|---|---|
| Example | 5 | 15 | 30 | 45 |
| Example 1 | 84 | 92 | 95 | 95 |
| (% Dissolution) | | | | |
| Example 2 | 86 | 94 | 96 | 97 |
| (% Dissolution) | | | | |
| Example 3 | 91 | 97 | 98 | 99 |
| (% Dissolution) | | | | |

### Example 5-7

| | Exampl | Exampl | Exampl |
|---|---|---|---|
| Ingredients | Mg/tab | Mg/tab | Mg/tab |
| Drug dispersion | | | |
| Trametinib DMSO* | 2.26 | 2.26 | 2.26 |
| SEPICLEAR ™ ^{#} | 2.0 | 2.0 | 2.0 |
| Acetonitrile | q.s | q.s | — |
| Dichloromethane | q.s | — | q.s |
| Intragranular portion | | | |
| Microcrystalline | 52.74 | 52.74 | 52.74 |
| Mannitol | 102.0 | 102.0 | 102.0 |
| Poloxamer | 1.0 | 1.0 | 1.0 |
| Croscarmellose | 2.0 | 2.0 | 2.0 |
| Extra granular | | | |
| Microcrystalline | 10.0 | 10.0 | 10.0 |
| Croscarmellose | 2.0 | 2.0 | 2.0 |
| Magnesium stearate | 1.0 | 1.0 | 1.0 |
| Total tablet weight | 175 | 175 | 175 |
| Film-coating (2.4%) | 4.37 | 4.37 | 4.37 |
| Total tablet weight | 179.4 | 179.4 | 179.4 |

*2.26 mg of trametinib DMSO is equivalent to 2 mg of trametinib.
^{#}SEPICLEAR ™ is a pure lipoamino acid.

Brief Manufacturing Process:

a) Trametinib DMSO and SEPICLEAR™ were dispersed in acetonitrile and/or dichloromethane,

b) intragranular excipients were sifted, blended and the resultant blend was granulated with the dispersion of step (a),

c) the granules obtained were dried and sifted,

d) extragranular excipients were sifted separately and mixed with the dried granules obtained in step (c),

e) lubricated granules of step (d) were compressed into tablets using suitable tooling.

### Example 8-9

| | Exampl | Exampl |
|---|---|---|
| Ingredients | Mg/tab | Mg/tab |
| Trametinib DMSO* | 2.26 | 1.13 |
| Sodium lauryl | 0.4 | 0.4 |
| Colloidal silicon | 0.4 | 0.4 |
| Hypromellose | 0.5 | 0.5 |
| Microcrystalline | 64.44 | 65.57 |
| Mannitol | 104.0 | 104.0 |
| Croscarmellose | 2.00 | 2.00 |
| Magnesium stearate | 1.00 | 1.00 |
| Core tablet weight | 175 | 175 |
| Film-coating (2.8%) | 3.85 | 3.85 |
| Total tablet weight | 178.9 | 178.9 |

*2.26 mg of trametinib DMSO is equivalent to 2 mg of trametinib & l.13 mg of trametinib DMSO is equivalent to 1 mg of trametinib.

Brief Manufacturing Process:

a) Trametinib DMSO solvate was mixed with sodium lauryl sulfate, colloidal silicon dioxide, hypromellose and croscarmellose sodium,

b) microcrystalline cellulose and mannitol were sifted through #30 mesh,

c) step (a) and step (b) were mixed to get a uniform blend,

d) magnesium stearate was sifted through #40 mesh,

e) blend of step (c) was lubricated with the above sifted magnesium stearate,

f) lubricated blend of step (e) was compressed the into tablets using suitable tooling, and

g) finally, tablets obtained in step (f) were film coated using a suitable coating material.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

We claim:

1. A tablet composition comprising:
a) trametinib or its pharmaceutically acceptable salt or solvate, and
b) greater than or equal to 90 wt % of pharmaceutically acceptable excipients, based on the total weight of the composition.

2. The tablet composition of to claim 1, wherein trametinib is in the form of trametinib dimethyl sulfoxide.

3. The tablet composition of claim 1, wherein the pharmaceutically acceptable excipients are diluents, binders, disintegrants, solubility enhancers, lubricants, glidants, or a combination thereof.

4. The tablet composition of claim 1, wherein the excipients comprise a solubility enhancer that is lipoamino acid, a poloxamer or both, in an amount of about 0.1 wt % to about 10 wt %, based on the total weight of the composition.

5. The tablet composition of claim 1, further comprising a film coating.

6. A method of preparing a tablet composition, comprising
a) dispersing trametinib or its pharmaceutically acceptable salt or solvate in one or more non-aqueous solvents to provide a dispersion,
b) sifting and blending one or more intragranular excipients to provide a blend,
c) granulating the blend of step (b) using the dispersion of step (a) to provide granules,
d) drying the granules obtained in step (c) to provide dried granules,
e) sifting one or more extragranular excipients and blending the sifted extragranular excipients with the dried granules of step (d) to provide blended granules, and
f) compressing the blended granules of step (e) into tablets.

7. The method of claim 6, wherein non-aqueous solvent is acetonitrile, dichloromethane, ethanol, methanol, acetaldehyde, acetone, benzene, 1,2-dichloroethane, N,N-dimethylformamide, 1,4-dioxane, epichlorhydrin, ethyl acetate, ethyl ether, ethylene glycol, 2-ethoxyethanol (acetate), formaldehyde, isopropanol, methyl n-butyl ketone, methyl ethyl ketone, 2-methoxyethanol (acetate), perchloroethylene, toluene, 1,1,1-trichloroethane, trichloroethylene, or a combination thereof.

8. The product of the process of claim 6.

9. A method of treating unresectable or metastatic melanoma with BRAF V600E or V600K mutations in a patient in need thereof, comprising administering to the patient the composition according to claim 1.
